(19) United States
(12) Patent Application Publication
(10) Pub. No.: US 2022/0323272 A1
(43) Pub. Date: Oct. 13, 2022

(54) Stretchable Laminate, Locking Member, And Sanitary Material

(71) Applicant: 3M INNOVATIVE PROPERTIES COMPANY, St. Paul, MN (US)

(72) Inventors: Kioshi Kunihiro, Sagamihara (JP); Hiroyasu Nagata, Tokyo (JP); Hirofumi Hosokawa, Tokyo (JP)

(21) Appl. No.: 17/595,994

(22) PCT Filed: Jun. 16, 2020
(86) PCT No.: PCT/IB2020/055620

§ 371 Date: Dec. 1, 2021

(30) Foreign Application Priority Data
Jun. 21, 2019 (JP) 2019-115779

(51) Int. Cl. A61F 13/58; B32B 5/02; B32B 27/12; B32B 7/12; B32B 27/08

(52) U.S. Cl. CPC A61F 13/58; B32B 5/022; B32B 27/12; B32B 5/026; B32B 7/12; B32B 27/08; B32B 5/024; B32B 2307/51; B32B 2255/02; B32B 2307/72; B32B 2555/02

(57) ABSTRACT

To provide a stretchable laminate, a locking member, and a sanitary material that can be easily extended at a low stress as well as can enhance the strength. A stretchable laminate 10 according to one embodiment includes a non-woven fabric 14, an elastomer layer 13 laminated on the non-woven fabric 14, and a knitted fabric 15 laminated on the elastomer layer 13. A locking member according to one embodiment is a locking member that is attached to a sanitary material, the locking member including a stretchable laminate 10 including a knitted fabric 15 laminated on an elastomer layer 13, and an engaging portion provided on an end portion of the stretchable laminate 10 opposite to the sanitary material.

### TECHNICAL FIELD

One aspect of the present disclosure relates to a stretchable laminate, a locking member, and a sanitary material.

### BACKGROUND

JP 2009-132081A describes a stretchable laminate and an article using the same. The stretchable laminate includes a first non-woven fabric, a second non-woven fabric, and a layer provided between the first non-woven fabric and the second non-woven fabric, the layer including an elastomer composition. In the stretchable laminate, the layer including an elastomer composition has an elongation at break greater than or equal to 115%. A stretchable region is provided in the center of the stretchable laminate, and a non-stretchable region is provided on each of both ends of the stretchable laminate.

The stretchable laminate is attached to the disposable diaper, and one of the pair of non-stretchable regions is attached to the disposable diaper. A mechanical fastener is attached to the other of the pair of non-stretchable regions. The stretchable laminate is used as an ear portion of the disposable diaper. It is supposed that, in this stretchable laminate, a reduction in elastic force is small during repetitive extension and contraction and delamination of the non-woven fabric and tearing of the layer including the elastomer composition do not occur.

### SUMMARY

### Technical Problem

Incidentally, in a stretchable laminate including a layer including an elastomer composition, the stretchable laminate may be extended when locked with a mechanical fastener and is required to have strength to withstand when extended. Further in some cases, a large stress also may be required in order to extend the stretchable laminate. Accordingly, the stretchable laminate is required to be easily extended at a low stress and to enhance its strength.

### Solution to Problem

The stretchable laminate according to one aspect of the present disclosure includes a non-woven fabric, an elastomer layer laminated on the non-woven fabric, and a knitted fabric laminated on the elastomer layer.

In the stretchable laminate according to one aspect described above, the knitted fabric is a reticulated member formed by formation of loops by a plurality of yarns as well as entanglement of the plurality of yarns with one another. Therefore, lamination of the knitted fabric on the elastomer layer enables the stretchable laminate to be easily extended at a low stress as well as entanglement of the yarns of the knitted fabric with one another in stretching enables the strength in stretching to be enhanced. Accordingly, this stretchable laminate can be easily extended at a low stress as well as its strength can be enhanced.

The stretchable laminate may include an adhesive layer interposed between the knitted fabric and the elastomer layer.

The adhesive layer may be made of a pressure sensitive adhesive.

The adhesive layer may include an anchor coating material.

The adhesive layer may be a film layer made by including a resin.

The density of the knitted fabric in the course direction may be greater than or equal to 15 yarns/inch and less than or equal to 50 yarns/inch.

The density of the knitted fabric in the wale direction may be greater than or equal to 15 yarns/inch and less than or equal to 50 yarns/inch.

The elongation ratio of the knitted fabric during elongation in at least one direction may be greater than or equal to 150%.

A stretchable laminate according to another aspect of the present disclosure includes a non-woven fabric, an elastomer layer laminated on the non-woven fabric, and a woven fabric laminated on the elastomer layer, and at least a portion of a plurality of yarns composing the woven fabric is bonding-processed.

The stretchable laminate described above includes a woven fabric laminated on an elastomer, and at least a portion of a plurality of yarns composing the woven fabric is bonding-processed. Thus, lamination of the woven fabric, in which at least a portion of the plurality of yarns is bonding-processed, on the elastomer layer enables the stretchable laminate to be easily extended at a low stress as well as bonding-processing at least a portion of the yarns of the woven fabric enables the strength in stretching to be enhanced. Accordingly, the stretchable laminate can be easily extended at a low stress as well as can enhance the strength.

A locking member according to one aspect of the present disclosure is a locking member that is configured to be attached to a sanitary material. The locking member includes a stretchable laminate including a knitted fabric laminated on an elastomer layer, and a locking portion provided on an end portion of the stretchable laminate opposite to the sanitary material.

This locking member includes a stretchable laminate including a knitted fabric attached to the sanitary material as well as laminated on the elastomer layer. A locking portion is provided on an end portion of the stretchable laminate opposite to the sanitary material. Accordingly, the locking member attached to the sanitary material can be easily extended at a low stress and the locking portion in the sanitary material can be easily locked by the fact that the locking member attached to the sanitary material includes the stretchable laminate including the knitted fabric. Further, the locking member includes the stretchable laminate including the knitted fabric and thus can enhance the strength of the locking member. Accordingly, breakage and the like of the locking member in stretching can be more reliably suppressed.

The stretchable laminate may include a pair of low-stretchable portions, one of the pair of low-stretchable portions may be attached to a sanitary material, and the locking portion may be provided on the other of the pair of low-stretchable portions.

The above-mentioned locking member includes a pair of non-woven fabrics, and the stretchable laminate includes a pair of low-stretchable portions respectively attached to each of the pair of non-woven fabrics. One of the pair of non-woven fabrics may be attached to a sanitary material, and the locking portion may be provided on the other of the pair of non-woven fabrics.

A locking member according to another aspect of the present disclosure is a locking member that is configured to be attached to a sanitary material. The locking member includes a stretchable laminate being laminated on an elastomer layer and including a woven fabric, at least a portion of a plurality of yarns of the woven fabric being bonding-processed, and a locking portion provided on an end portion of the stretchable laminate opposite to the sanitary material.

The above-mentioned locking member includes a stretchable laminate, and the stretchable laminate includes a woven fabric laminated on an elastomer layer. Accordingly, the locking member attached to the sanitary material can be easily extended at a low stress by the fact that the locking member includes the stretchable laminate including the woven fabric, and the locking portion in the sanitary material can be easily locked. Further, in the woven fabric in the stretchable laminate for the locking member, at least a portion of the plurality of yarns is bonding-processed to thereby enable the strength of the locking member to be enhanced. Accordingly, breakage and the like of the locking member in stretching can be more reliably suppressed.

A sanitary material according to one aspect of the present disclosure includes the above-mentioned locking member as a tab. Since including the above-mentioned locking member as a tab, this sanitary material can easily be extended at a low stress as well as can enhance its strength. Accordingly, the tab can be extended to facilitate locking as well as can have a high strength.

### Advantageous Effects of Invention

According to one aspect of the present disclosure, the stretchable laminate, the locking member, and the sanitary material can be easily extended at a low stress as well as can enhance the strength.

### BRIEF DESCRIPTION OF THE DRAWINGS

FIG. 1 is a view schematically illustrating a sanitary material including a locking member according to an embodiment.

FIGS. 2(a) and 2(b) are cross sectional views illustrating various examples of a locking member and a sanitary material including a stretchable laminate according to an embodiment.

FIG. 3 is a cross sectional view illustrating a stretchable laminate according to an embodiment.

FIG. 4 is a view schematically illustrating an example of the knitted fabric of the stretchable laminate of FIG. 3.

FIG. 5 is a view illustrating an example of a state where the knitted fabric of FIG. 4 is extended.

FIG. 6 is a view illustrating an example of a method for manufacturing the stretchable laminate of FIG. 3.

FIG. 7 is a view schematically illustrating the woven fabric of a stretchable laminate according to a modified example.

FIG. 8 is a view schematically illustrating a sanitary material and a locking member according to a modified example.

FIG. 9 is a graph showing the experimental results of the stretchable laminates according to the examples.

FIG. 10 is a graph showing the experimental results of the stretchable laminates according to examples.

FIG. 11 is a graph showing the experimental results of the stretchable laminates according to the examples.

### DETAILED DESCRIPTION

Hereinafter, embodiments for implementing a stretchable laminate, a locking member, and a sanitary material according to the present disclosure will be described in detail with reference to the accompanying drawings. In the description of the drawings, the same or corresponding elements are denoted by the same reference numerals, and overlapping descriptions are omitted appropriately. In addition, the drawings may be partially simplified or exaggerated for ease of understanding, and the dimensional ratios, etc. are not limited to those illustrated in the drawings.

A “non-woven fabric” herein indicates a fabric formed by bonding or entangling fibers by a mechanical action or a chemical action. A “stretchable laminate” is a laminate including an elastomer layer having stretchability. The “stretchable laminate” can be used, for example, in applications in which excellent stretching is necessary, as an example, a portion of a sanitary material such as a diaper. The “stretchable laminate” can be used, for example, in applications in which aesthetics are required and applications in which excellent stretchability (skin feel or the like) is required.

At least either one of a woven fabric and a knitted fabric is laminated on the elastomer layer. A “woven fabric” indicates a fabric formed with warp yarns and weft yarns intersecting one another, and a “knitted fabric” indicates a fabric (knit) knitted by continuously forming loops formed in yarns. The “knitted fabric” at least partially includes loops where a plurality of yarns is entangled with one another (closed loops). “Sanitary material” is used to maintain sanitary condition, including, for example, disposable equipment.

“Sanitary material” includes, for example, absorbent articles such as disposable diapers, sanitary napkins, and incontinence pads, as well as sanitary articles such as hemostatic tapes. A “locking member” is attached to the “sanitary material”. The “locking member” and “locking portion” respectively indicate a member and a portion that are locked to a certain object. “Lock” indicates that an object is attached to another object, and includes a state where two objects are fastened and fixed.

The stretchable laminate may include a low-stretchable portion, for example, and be attached to the sanitary material by interposing the low-stretchable portion. The “low-stretchable portion” indicates a portion having lower stretchability than that of portions other than the low-stretchable portion, and includes a site of which stretchability is suppressed by embossing (non-stretchable portion).

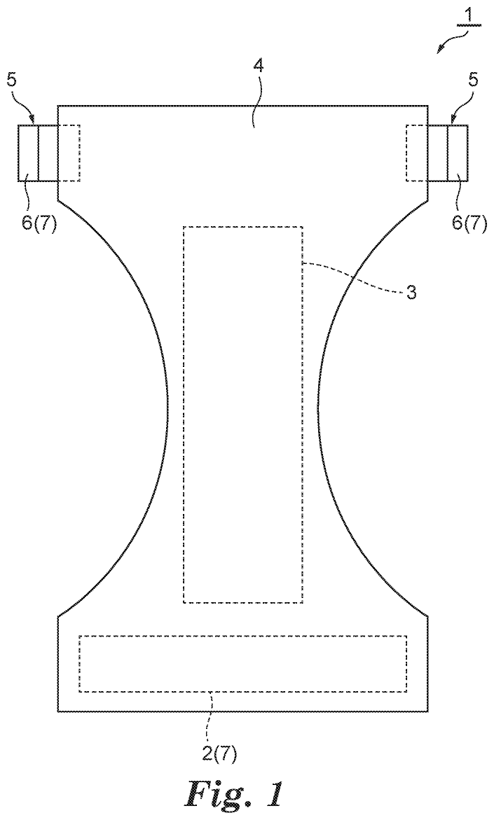

FIG. 1 is a view schematically illustrating a sanitary material 1 according to the embodiment. FIG. 1 illustrates a sanitary material 1 which is an open type (tape type) diaper as an example. However, the sanitary material 1 as a diaper may be a pad type, pants type, or the like, and the type of the diaper may be appropriately changed. As an example, the sanitary material 1 is a disposable diaper. The sanitary material 1 includes a body portion 4 including, for example, an absorbent body 3 that absorbs liquid. As an example, the body portion 4 is constituted by a non-woven fabric.

The sanitary material 1 is a tab type diaper, and the width of the body portion 4 becomes narrower toward the center of the longitudinal direction of the body portion 4 (longitudinal direction of the absorbent body 3). That is, the width of the body portion 4 is wider at both ends of the longitudinal direction of the body portion 4. An engaging portion 2 is provided on one end of the longitudinal direction of the body portion 4, and tabs 5 (tab members) that engage with the engaging portion 2 are provided on the other end of the longitudinal direction of the body portion 4. The tab 5 is a pinch portion to be attached to the sanitary material 1 and serves as a locking member to be locked to the sanitary material 1. The tabs 5 are each attached to both ends of the width direction of the body portion 4. As an example, the tabs 5 are each formed into a rectangular shape.

The tabs 5 are portions that are stretched so as to wrap around the waist of a wearing target (such as an infant, an elderly person, or the like) when the sanitary material 1 is worn on the wearing target. Each of the tabs 5 includes an engaging portion 6, and a mechanical fastener 7 is formed by the engaging portion 6 of the tab 5 and the engaging portion 2 described above. The mechanical fastener 7, which includes, for example, a plane fastener or a structure called a hook-and-loop fastener, is a fastener planarly detachable.

The engaging portion 2 is provided, for example, on the front surface of the region covering the lower belly of a wearing target. When the sanitary material 1 is worn on a wearing target, at least either of the pair of right and left tabs 5 is stretched in the width direction of the body portion 4 so as to cause the body portion 4 to wrap around the waist of the wearing target. Each tab 5 is stretched by an appropriate stretching force, and the engaging portions 6 of the tabs 5 are affixed to the engaging portion 2 of the body portion 4 and fixed.

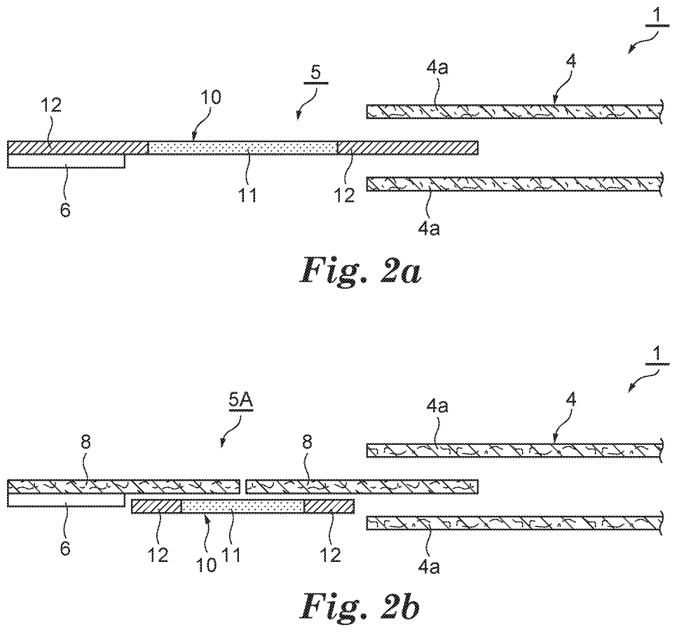

FIG. 2(a) is an enlarged cross-sectional view of the sanitary material 1 and the tab 5. The tab 5 includes the above-mentioned engaging portion 6 and a stretchable laminate 10 that has been processed into a predetermined shape. The stretchable laminate 10 includes a stretchable portion 11 and a low-stretchable portion 12. For example, a pair of low-stretchable portions 12 are each provided on both the sides of the stretchable portion 11. However, the low-stretchable portion 12 may be provided on one side of the stretchable portion 11, and the number and aspect of arrangement of the low-stretchable portion 12 can be appropriately changed.

The low-stretchable portion 12 is a stretch regulating region that includes a plurality of crimping portions (concave-convex patterns) arranged in dots, for example, and in which the stretchability is reduced by the plurality of crimping portions. As a specific example, the crimping portions of the low-stretchable portion 12 are formed by embossing performed with a nip roll including dot-shaped protrusions on the surface thereof.

The larger the area occupied by the crimping portions, the lower the stretchability of the low-stretchable portion 12, and the stretchability of the low-stretchable portion 12 can be adjusted depending on the aspect of the crimping portions. Note that the crimping portions of the low-stretchable portion 12 may be formed on each of both the surfaces of the stretchable laminate 10, or may be formed on one surface of the stretchable laminate 10. Alternatively, the low-stretchable portion 12 may not have a concave-convex pattern of the crimping portions and may be crimped in the entire region.

The stretchable laminate 10 includes, for example, a pair of low-stretchable portions 12 each provided on both the sides of the stretchable portion 11, for example. The engaging portion 6 is fixed to one of the low-stretchable portions 12 so as to superpose on the low-stretchable portion 12. The other low-stretchable portion 12 is fixed to the sanitary material 1 (body portion 4). For example, the other low-stretchable portion 12 is in a state of being sandwiched between a pair of non-woven fabrics 4a that constitute the body portion 4, and fixed to the sanitary material 1.

The stretchable portion 11 is located between the pair of low-stretchable portions 12. In the stretchable laminate 10, the sanitary material 1 and the engaging portion 6 are fixed in each of the pair of low-stretchable portions 12 having a low stretchability and a high shape stability. Additionally, since the stretchable portion 11 between the pair of low-stretchable portions 12 is stretchable, the stretchable portion 11 is extended to thereby to facilitate the attachment of the engaging portion 6 to the engaging portion 2 of the body portion 4. As a result, stable affixing is enabled.

FIG. 2(b) illustrates the tab 5A according to a modified example. The tab 5A includes an engaging portion 6, a stretchable laminate 10, and a non-woven fabric 8. The tab 5A includes two sheets of non-woven fabrics 8, for example, and the two non-woven fabrics 8 are connected to each other by the stretchable laminate 10. One low-stretchable portion 12 of the stretchable laminate 10 is fixed to one of the two non-woven fabrics 8, and the other low-stretchable portion 12 of the stretchable laminate 10 is fixed to the other of the two non-woven fabrics 8.

The stretchable portion 11 of the stretchable laminate 10 is located between the pair of non-woven fabrics 8, and the distance between the pair of non-woven fabrics 8 changes due to stretching of the stretchable portion 11. The engaging portion 6 is fixed to one of the two non-woven fabrics 8 so as to be superposed on the non-woven fabric 8, and the other of the two non-woven fabrics 8 is fixed to the sanitary material 1. The other non-woven fabric 8 may be in a state of being sandwiched between the pair of non-woven fabrics 4a of the body portion 4, and may be fixed to the sanitary material 1. In this manner, it is also possible to fix the stretchable laminate 10 and the engaging portion 6 to the sanitary material 1 by interposing the non-woven fabric 8.

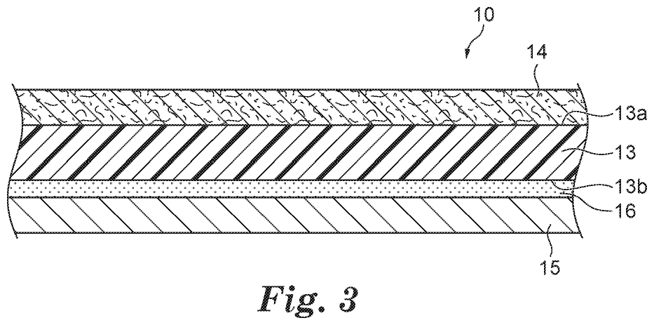

FIG. 3 is a cross-sectional view schematically illustrating the laminate structure of the stretchable laminate 10. As shown in FIG. 3, the stretchable laminate 10 includes an elastomer layer 13, a non-woven fabric 14 laminated on one main surface 13a of the elastomer layer 13, and a knitted fabric 15 laminated on the other main surface 13b of the elastomer layer 13. When the direction in which the stretchable laminate 10 is sent out in a state where the non-woven fabric 14 is laminated thereon is designated as a Machine Direction MD, and the direction orthogonal to the MD is designated as a Cross Direction CD, the stretchability in the CD is higher than that in the MD of the stretchable laminate 10 according to the present embodiment. For example, the CD corresponds to the direction in which the low-stretchable portion 12 and the stretchable portion 11 are aligned (the left and right direction in FIGS. 1 and 2).

The elastomer layer 13 is, for example, a stretchable film formed from a resin material containing an elastomer. The type of elastomer of the elastomer layer 13 is not particularly limited, and examples thereof include styrene-isoprene-styrene block copolymers (SIS), styrene-butadiene-styrene block copolymers (SBS), styrene-ethylene/butylene-styrene block copolymers (SEBS), polyurethane, ethylene copolymers (e.g., ethylene-vinyl acetate, ethylene-propylene copolymers, or ethylene-propylene-diene terpolymers), or propylene oxide (PO). In view of the convenience of the manufacturing process, the elastomer layer 13 may include a multilayer structure including a skin layer such as a polyolefin on at least one main surface of the core layer of the elastomer.

The resin material that composes the elastomer layer 13 may contain other materials besides those described above. For example, the resin material of the elastomer layer 13 may contain a stiffening agent (e.g., polyvinyl styrene, polystyrene, poly α-methyl styrene, polyester, epoxy resin, polyolefin, or coumarone-indene resin), a viscosity reducing agent, a plasticizer, a tackifier (e.g., an aliphatic hydrocarbon tackifier, an aromatic hydrocarbon tackifier, a terpene resin tackifier, a hydrogenated terpene resin tackifier), a dye, a pigment, an antioxidant, an antistatic agent, an adhesive, an antiblocking agent, a slip agent, a heat stabilizer, a light stabilizer, a blowing agent, glass bubbles, starch, a metal salt, or microfibers, or the like.

The non-woven fabric 14 is formed from a material that can be processed as a fiber. The non-woven fabric 14 can be adjusted to have a desired softness by appropriate combination of materials and adjustment of the fiber length or the fiber thickness. The material of the non-woven fabric 14 includes, for example, polypropylene fiber, mixed fiber of polyester fiber and polyolefin fiber, or concentric type composite fiber of polyethylene terephthalate core covered by polyethylene. The non-woven fabric 14 may be prepared by spun-bonding, spunlacing, or thermal bonding, for example.

An adhesive layer 16, for example, is interposed between the elastomer layer 13 and the knitted fabric 15, and the knitted fabric 15 may be bonded to the elastomer layer 13 by the adhesive layer 16. The adhesive layer 16 is, for example, a pressure sensitive adhesive, and may be a hot melt pressure sensitive adhesive. The adhesive layer 16 may be an anchor coating agent, and in this case, the knitted fabric 15 to which the anchor coating material is applied and the elastomer layer 13 are bonded to each other. The adhesive layer 16 may be a pre-formed film layer. For example, a film in a molten state may be affixed between the elastomer layer 13 and the knitted fabric 15. Further, the adhesive layer 16 may be a welded portion formed by welding, and the aspect of the adhesive layer 16 can be appropriately changed. Furthermore, the adhesive layer 16 can be omitted.

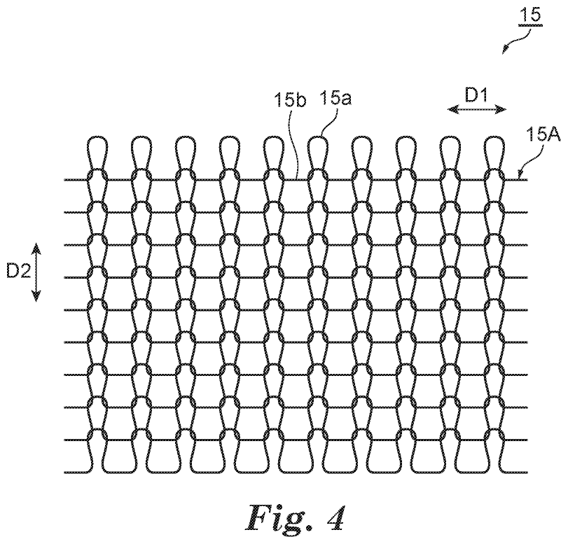

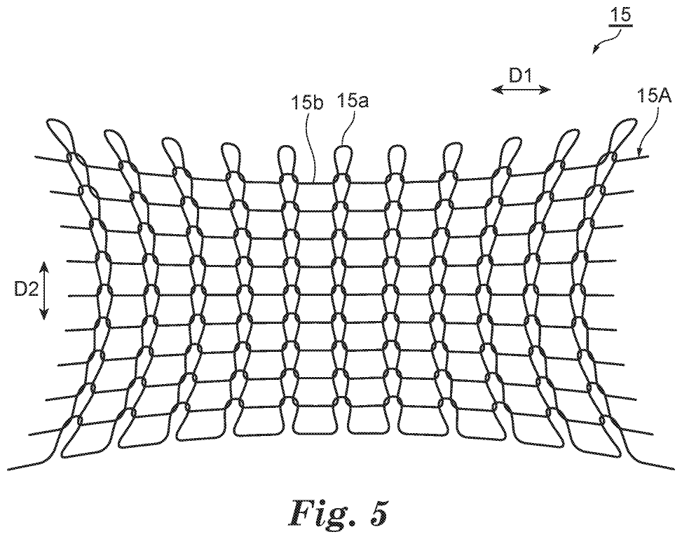

FIGS. 4 and 5 are views illustrating the knitted fabric 15 as an example. The knitted fabric 15 includes, for example, a plurality of yarns 15A forming knitting yarns. Each of the yarns 15A forms a plurality of loops 15a each protruding in a ring shape from the straight portion 15b, the plurality of loops 15a are continuously formed, and the plurality of yarns 15A are entangled with each other in the loops 15a to thereby form the knitted fabric 15. As an example, the knitted fabric 15 may be a knitted fabric composed of warp yarns, weft yarns, and loop yarns, or, as shown in FIGS. 4 and 5, may be formed by weft knitting, in which the loops 15a form the knitted fabric 15 while advancing in the course direction D1 (lateral direction). For example, the knitted fabric 15 may be a tricot pile-knitted fabric, which is knitted by a two-bar knitting machine.

When the knitted fabric 15 is formed by weft knitting, the stretchability of the knitted fabric 15 can be further enhanced. Examples of the weft knitting include flat stitch, rubber stitch, pearl stitch, tuck stitch, float stitch, lace stitch, full cardigan stitch, half cardigan stitch, pelerine stitch, eyelet stitch, interlock stitch, multi-process interlock stitch, double jersey stitch, racked stitch, broad rib stitch, napped stitch, fleecy stitch, plating stitch, embroidery plating stitch, Rahben stitch, twist stitch, or argyle stitch.

However, the knitted fabric 15 may be formed by warp knitting, in which the loops are continuously knitted in the wale direction D2. In this case, it is possible to make the fabric more unlikely to be unknitted and to prevent a run more reliably. Examples of the warp knitting include single denbigh stitch, single vandyke stitch, rib stitch, single cord stitch, Berlin stitch, two needle stitch, shell stitch, double denbigh stitch, atlas stitch, cord stitch, russel stitch, tricot stitch, half tricot stitch, satin stitch, sharkskin stitch, queen's coat stitch, milanese stitch, idle swing stitch, elastic stitch, pile stitch, tuck stitch, figured stitch, pineapple stitch, fleecy knitted stitch, fleecy atlas stitch, fleecy coat stitch, or lace stitch.

The material of the yarn 15A composing the knitted fabric 15 is not particularly limited, and examples thereof include polypropylene, polyester, polyethylene, polyamide, polyurethane, rayon, copolymers or mixtures thereof, or natural fibers. Further, in the material of the yarns 15A, polyamide may be included in order to improve the strength, or, in terms of material cost and environmental stability, polyester may be included. The yarns 15A of the knitted fabric 15 may have a monofilament structure or may have a multifilament structure.

The density of the knitted fabric 15 in the course direction D1 is, for example, is greater than or equal to 15 yarns/inch and less than or equal to 50 yarns/inch. Here, the yarns/inch is a unit indicating the number of yarns 15A per inch as a density. The lower limit of the density in the course direction D1 may be 20 yarns/inch, 25 yarns/inch, or 30 yarns/inch. The upper limit of the density in the course direction D1 may be 45 yarns/inch, 40 yarns/inch, or 35 yarns/inch.

The density of the wale direction D2 of the knitted fabric 15 is, for example, is greater than or equal to 15 yarns/inch and less than or equal to 50 yarns/inch, as the density in the course direction D1, for example. In addition, the lower limit of the density in the wale direction D2 may be 20 yarns/inch, 25 yarns/inch, or 30 yarns/inch. The upper limit of the density in the wale direction D2 may be 45 yarns/inch, 40 yarns/inch, or 35 yarns/inch.

For example, the knitted fabric 15 hardly extends in the MD (direction in which the stretchable laminate 10 is sent out), but extends in the CD (direction orthogonal to the MD). For example, the knitted fabric 15 may extend by greater than or equal to 150% (less than or equal to 250% by way of example) when the knitted fabric 15 having a length of 15 mm is stretched at 3 N/35 mm (width) as compared with before stretched. The tensile strength of the knitted fabric 15 is, for example, greater than or equal to 12 N/35 mm, greater than or equal to 14 N/35 mm, or greater than or equal to 20 N/35 mm. In addition, the knitted fabric 15 has a width reduction amount greater than or equal to 15 mm (or greater than or equal to 20 mm and less than or equal to 32 mm) when the knitted fabric 15 having a width of 35 mm in a unstretched state is stretched in the length direction (CD).

Next, one example of a method of manufacturing the stretchable laminate 10 will be described. As an example, the stretchable laminate 10 is manufactured by a simultaneous melt-extrusion lamination method. In an exemplary steps of the simultaneous melt-extrusion lamination method, conducted are a “Laminate step” of laminating the non-woven fabric 14 and the knitted fabric 15 on the elastomer layer 13 to manufacture the stretchable laminate 10, an “Activate step” of subjecting the stretchable laminate 10 to an elongation treatment, and a “Slitting step” of forming a slit for conveyance in the stretchable laminate 10.

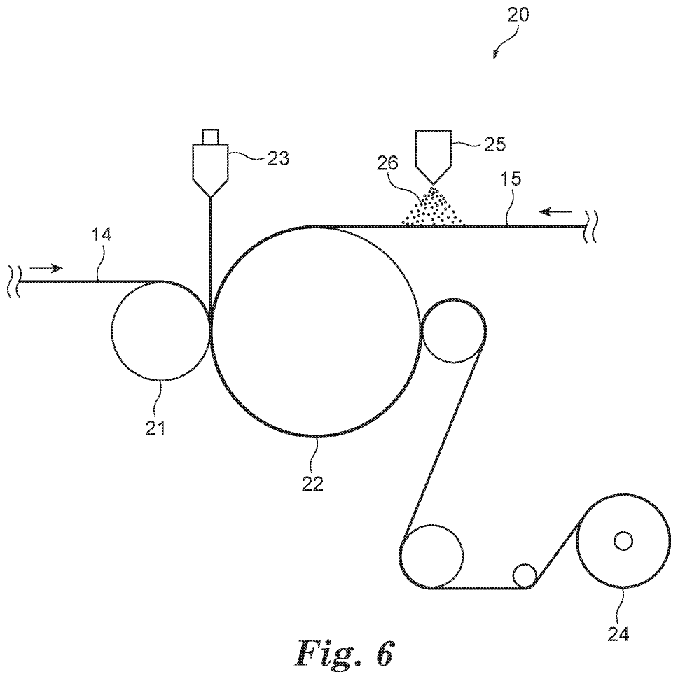

As shown in FIG. 6, in the “Laminate step”, a stretchable laminate 10 is manufactured by, for example, a melt-extrusion lamination apparatus 20. The melt-extrusion lamination apparatus 20 includes a nip roll 21 and a cooling roll 22 disposed so as to face the nip roll 21, by way of an example. The nip roll 21 may include, for example, a convex portion that forms the crimping portion of the low-stretchable portion 12 of the stretchable laminate 10. The cooling roll 22 may be a calendar roll including a smooth surface. The melt-extrusion lamination apparatus 20 may further include a die 23 between the nip roll 21 and the cooling roll 22 to supply the elastomer layer 13. The die 23 is a T-die, for example.

The elastomer layer 13 is sent out, for example, in a molten state from the die 23 and guided between the nip roll 21 and the cooling roll 22. The non-woven fabric 14 is conveyed to the nip roll 21, and the knitted fabric 15 is conveyed to the cooling roll 22. The elastomer layer 13, the non-woven fabric 14, and the knitted fabric 15 meet between the nip roll 21 and the cooling roll 22, and the elastomer layer 13 sent out from the die 23 is sandwiched between the non-woven fabric 14 and the knitted fabric 15 at the point of the meeting. The stretchable laminate 10 including the non-woven fabric 14 and the knitted fabric 15 with the elastomer layer 13 sandwiched therebetween is cooled by the cooling roll 22, and the elastomer layer 13 is solidified due to this cooling.

An application gun 25 configured to apply a hot melt adhesive 26 to the knitted fabric 15 may be provided on upstream of the cooling roll 22 in the conveyance path for the knitted fabric 15. In this case, on the knitted fabric 15 before laminated on the elastomer layer 13, the hot melt adhesive 26 is applied from the application gun 25. Then, the hot melt adhesive 26 is sandwiched between the elastomer layer 13 and the knitted fabric 15 between the nip roll 21 and the cooling roll 22. Solidification of the sandwiched hot melt adhesive 26 causes an adhesive layer 16 to be formed between the elastomer layer 13 and the knitted fabric 15. The stretchable laminate 10 discharged from the nip roll 21 and the cooling roll 22 is conveyed by a conveyance roll to a winding roll 24 and rolled up and held by the winding roll 24. This completes the “Laminate step” of the stretchable laminate 10.

After the “Laminate step”, the “Activate step” is conducted. In the “Activate step”, in order to reduce the initial strength of the non-woven fabric to thereby reduce the stress, the stretchable laminate 10 is subjected to an elongation treatment in the CD (Cross Direction). The non-woven fabric 14 laminated on the elastomer layer 13 is deformed by execution of the “Activate step” after the “Laminate step”. This deformation contributes to reducing the stress of the stretchable laminate 10. After the “Activate step”, the “Slitting step” is conducted. In the “Slitting step”, for example, a wide stretchable laminate 10 is subjected to a cutting treatment so that the stretchable laminate 10 has a desired width. Through this “Slitting step”, a series of steps of manufacturing the stretchable laminate 10 is completed.

Next, the action and effect of the stretchable laminate, the locking member, and the sanitary material according to the present embodiment will be described.

In the stretchable laminate 10, the knitted fabric 15 is a reticulated member formed by formation of loops 15a by a plurality of yarns 15A as well as entanglement of the plurality of yarns 15A with one another. Therefore, lamination of the knitted fabric 15 on the elastomer layer 13 enables the stretchable laminate 10 to be easily extended at a low stress as well as entanglement of the yarns 15A of the knitted fabric 15 with one another in stretching enables the strength in stretching to be enhanced. Accordingly, the stretchable laminate 10 can be stretched at a low stress as well as can enhance its strength.

The stretchable laminate 10 may include an adhesive layer 16 interposed between the knitted fabric 15 and the elastomer layer 13. In this case, since the adhesive layer 16 is interposed between the knitted fabric 15 and the elastomer layer 13, the binding of the knitted fabric 15 to the elastomer layer 13 can be enhanced. Thus, the strength of the stretchable laminate 10 can be further enhanced.

The adhesive layer 16 may be made of a pressure sensitive adhesive. In this case, adhesion of the knitted fabric 15 can be easily conducted by affixing the elastomer layer 13 to the knitted fabric 15 with a pressure sensitive adhesive.

The adhesive layer 16 may include an anchor coating material. In this case, the anchor coating material easily enables the knitted fabric 15 to adhere to the elastomer layer 13.

The adhesive layer 16 may be a film layer made by including a resin. In this case, a film made of a resin can be used as the adhesive layer 16.

The density of the knitted fabric 15 in the course direction D1 may be greater than or equal to 15 yarns/inch and less than or equal to 50 yarns/inch. In this case, when the density is greater than or equal to 15 yarns/inch, excessive stretching of the stretchable laminate 10 can be suppressed as well as the strength can be enhanced. Further, when the density is less than or equal to 50 yarns/inch, the stretchable laminate 10 can be extended at a low stress.

The density of the knitted fabric 15 in the wale direction D2 may be greater than or equal to 15 yarns/inch and less than or equal to 50 yarns/inch. In this case, as mentioned above, the strength can be enhanced as well as the stretchable laminate 10 can be extended at a low stress.

The elongation ratio of the knitted fabric 15 during elongation in at least one direction may be greater than or equal to 150%. In this case, the stretchable laminate 10 can be more easily extended at a low stress due to the elongation ratio of the knitted fabric 15 being greater than or equal to 150%.

When the elongation ratio of the stretchable laminate 10 is 50%, the tensile strength may be less than or equal to 4.0 N/25 mm, and the elongation ratio in stretching at 20 N/25 mm may be less than or equal to 250%. In this case, a low stress and a high strength can be achieved more significantly.

The locking members according to the present embodiment (e.g., the tabs 5) includes the stretchable laminate 10 including a knitted fabric 15 attached to the sanitary material 1 as well as laminated on the elastomer layer 13. An engaging portion 6 is provided on an end portion of the stretchable laminate 10 opposite to the sanitary material 1. Accordingly, the locking member attached to the sanitary material 1 can be extended at a low stress and the engaging portion 6 in the sanitary material 1 can be easily locked by the fact that the locking member attached to the sanitary material 1 includes the stretchable laminate 10 including the knitted fabric 15. Further, the locking member includes the stretchable laminate 10 including the knitted fabric 15 and thus can enhance the strength of the locking member. Accordingly, breakage and the like of the locking member in stretching can be more reliably suppressed.

The locking member may include a pair of low-stretchable portions 12, one of the pair of low-stretchable portions 12 may be attached to a sanitary material 1, and the other of the pair of low-stretchable portions 12 may be provided with an engaging portion 6. In this case, one of the low-stretchable portions 12 of the stretchable laminate 10 is attached to the sanitary material 1 as well as the engaging portion 6 is provided on the other low-stretchable portion 12 of the stretchable laminate 10. Thus, the engaging portion 6 can be attached to the sanitary material 1 through the stretchable laminate 10 by each of the pair of low-stretchable portions 12.

The locking member includes a pair of non-woven fabrics 8, and the stretchable laminate 10 includes a pair of low-stretchable portions 12 attached to each of the pair of non-woven fabrics 8. One of the pair of non-woven fabrics 8 is attached to a sanitary material 1, and the engaging portion 6 may be provided on the other of the pair of non-woven fabrics 8. In this case, one of the low-stretchable portions 12 of the stretchable laminate 10 is attached to one of the non-woven fabrics 8, and the other low-stretchable portion 12 of the stretchable laminate 10 is attached to the other non-woven fabric 8. Thus, the pair of non-woven fabrics 8 can be connected to each other through the stretchable laminate 10. Further, one of the non-woven fabrics 8 is attached to the sanitary material 1 as well as the engaging portion 6 is provided on the other of the non-woven fabrics 8. Accordingly, the engaging portion 6 can be attached to the sanitary material 1 by each of the pair of non-woven fabrics 8 provided on both the sides of the stretchable laminate 10.

The sanitary material 1 includes the locking members mentioned above as the tabs 5. Since the sanitary material 1 includes the locking members mentioned above as the tabs 5, this sanitary material can be extended at a low stress as well as can enhance its strength. Accordingly, the tabs 5 can be extended to facilitate locking as well as can have a high strength. Further, the tabs 5 are smaller than the tabs 45 of an ear type sanitary material 41 to be mentioned below, but even the small tabs 5 can exhibit high stretchability and a high strength. Accordingly, the tabs 5 of the tab type sanitary material 1 can exhibit significant stretchability and strength.

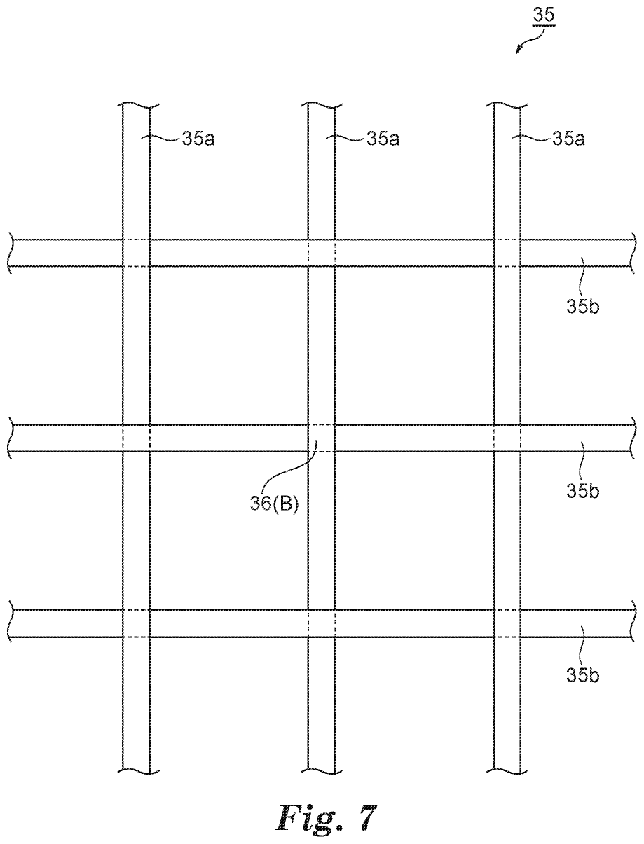

Next, a stretchable laminate and a locking member according to a modified example will be described. The stretchable laminate according to the modified example includes a woven fabric 35 instead of the knitted fabric 15, as illustrated in FIG. 7, for example. The locking member according to the modified example includes a stretchable laminate according to the modified example instead of the stretchable laminate 10 mentioned above. The woven fabric 35 is laminated on the elastomer layer 13 mentioned above, and the stretchable laminate according to the modified example includes the elastomer layer 13, a non-woven fabric 14, and the woven fabric 35. Types of woven fabric 35 include plain weave, satin weave, or twill weave. The woven fabric 35 may also be a chemical fiber textile (recycled fiber textile), a synthetic textile, a cotton textile, a wool textile, a filament textile, a silk textile, or a blended yarn textile thereof. In other words, the material of the yarns composing the woven fabric 35 can be changed as appropriate.

At least a portion of the plurality of yarns composing the woven fabric 35 are bonding-processed. As a specific example, the woven fabric 35 is formed by a plurality of warp yarns 35a and a plurality of weft yarns 35b intersecting each of the plurality of warp yarns 35a, and including a plurality of intersections 36 where the warp yarns 35a and the weft yarns 35b intersect one another. For example, at least a portion of the plurality of intersections 36 are bonding-processed portions B as the result of the bonding processing.

The stretchable laminate according to the modified example includes a woven fabric 35 laminated on the elastomer layer 13, and at least a portion of the yarns composing the woven fabric 35 is bonding-processed. Thus, since the woven fabric 35 in which at least a portion of the plurality of yarns is bonding-processed is laminated on the elastomer layer 13, the stretchable laminate can be easily extended at a low stress. Since at least a portion of the yarns of the woven fabric 35 is the bonding-processed portions B, the strength in stretching can be enhanced. Accordingly, the stretchable laminate can be extended at a low stress as well as can enhance its strength.

The locking member according to the modified example includes the stretchable laminate according to the modified example, and the stretchable laminate includes the woven fabric 35 laminated on the elastomer layer 13. Accordingly, the locking member attached to the sanitary material can be extended at a low stress by the fact that the locking member includes the stretchable laminate including the woven fabric 35, and thus the engaging portion in the sanitary material can be easily locked. Further, in the woven fabric 35 of the stretchable laminate of the locking member, at least a portion of the plurality of yarns is the bonding-processed portion B to thereby enable the strength of the locking member to be enhanced. Accordingly, breakage and the like of the locking member when the stretchable laminate is stretched can be more reliably suppressed.

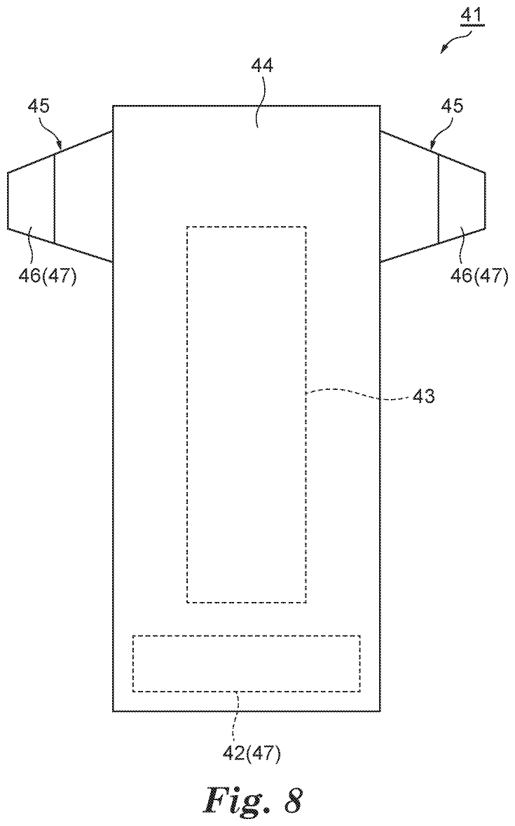

Next, a sanitary material 41 according to a modified example will be described with reference to FIG. 8. In the embodiment mentioned above, the sanitary material 1, which is a tab type diaper, was described, but the sanitary material 41 is an ear type diaper. In other words, the shape of the body portion 44 of the sanitary material 41 is a rectangular shape including long sides in the longitudinal direction of the absorbent body 43, and the width of the body portion 44 is constant regardless of the position of the body portion 44 in the longitudinal direction.

In the sanitary material 1, which is a tab type diaper, the width of the end portion in the longitudinal direction of the body portion 4 is wider, so that the tabs 5 can be relatively small. In contrast, the ear type sanitary material 41, in which the width of the body portion 44 is constant, includes relatively large tabs 45. The tabs 45 provided on the ear type sanitary material 41 may be referred to as ear portions (or ear members).

An engaging portion 42 is provided at one end in the longitudinal direction of the body portion 44, and tabs 45 to engage with the engaging portion 42 are provided on the other end of the longitudinal direction of the body portion 44. The configuration of the engaging portion 42, the absorbent body 43, the body portion 44, and the tabs 45 is the same as the configuration of the engaging portion 2, the absorbent body 3, the body portion 4, and the tabs 5 mentioned above, but the shape of the body portion 44 and the tabs 45 is different from the shape of the body portion 4 and the tabs 5.

In other words, as mentioned above, the body portion 44 has a rectangular shape, and the tabs 45 each have a trapezoidal shape that widens toward the joint with the body portion 44. The tabs 45 each includes, for example, an engaging portion 46 similar to the engaging portion 6 mentioned above and the stretchable laminate 10 mentioned above. A mechanical fastener 47 is formed by the engaging portion 46 of the tabs 45 and the engaging portion 42 of the body portion 44. The mechanical fastener 47 is, for example, a fastener similar to the mechanical fastener 7. As with the tabs 5, the tabs 45 of the sanitary material 41 configured as described above include the stretchable laminate 10 in which the knitted fabric 15 is laminated on the elastomer layer 13, and thus can be easily extended at a low stress as well as can enhance the strength.

The embodiment and modified example of the stretchable laminate, the locking member, and the sanitary material according to the present disclosure have been described hereinabove. However, the present invention is not limited to the aforementioned embodiment and modified example. The present invention can be variously modified without changing the gist of the claims. For example, the shape, size, number, material and the aspect of arrangement of each part constituting the stretchable laminate, the locking member, and the sanitary material can be appropriately changed.

### Examples

Next, examples of the stretchable laminate will be described. The present invention is not intended to be limited to the examples described below. In experiments according to the examples, a tensile test was performed on the stretchable laminates of Example 1 and Comparative Examples 1 to 5 mentioned below, and the tensile stress and the elongation ratio were measured. The tensile test was performed with a test piece width of 25 mm.

### Example 1

As illustrated in FIG. 3, the stretchable laminate according to Example 1 is a stretchable laminate 10 (tab 5) including a non-woven fabric 14 laminated on one main surface 13a of the elastomer layer 13 and a knitted fabric 15 laminated on the other main surface 13b of the elastomer layer 13 by interposing the adhesive layer 16. The stretchable laminate according to Example 1 was manufactured by conducting lamination on the elastomer layer 13 by affixing of melt extrusions. The non-woven fabric 14 used is a spunlace non-woven fabric of 32 grams per square meter (gsm), and the elastomer layer 13 used is an olefin-based elastomer of 28 gsm. The adhesive layer 16 used is a hot melt pressure sensitive adhesive of 7 gsm containing a styrene-based thermoplastic elastomer, and the knitted fabric 15 used is a warp-knitted (tricot knitted) polyester fiber (polyethylene terephthalate) of 29 gsm. The weight per unit area of the knitted fabric 15 (basis weight) is 24 g/m², the density of the knitted fabric 15 in the course direction is 21 yarns/inch, and the density of the knitted fabric 15 in the wale direction is 29 yarns/inch.

### Comparative Example 1

The stretchable laminate according to Comparative Example 1 is a stretchable laminate in which a non-woven fabric is laminated on both the main surfaces of the elastomer layer. The stretchable laminate according to Comparative Example 1 is an ear (large trapezoidal tab) for “Pampers Premium Care S size”, which are diapers manufactured by P&G Company. The stretchable laminate according to Comparative Example 1 was manufactured by laminating a spun-bonded non-woven fabric on both the main surfaces of the elastomer layer by lamination of a hot melt pressure sensitive adhesive. As the elastomer layer, a layer including a three-layer structure of skin layer-core layer-skin layer that has subjected to “activation” is used, and the hot melt pressure sensitive adhesive was applied by pattern coating.

### Comparative Example 2

The stretchable laminate according to Comparative Example 2 is a stretchable laminate in which a non-woven fabric is laminated on both the main surfaces of the elastomer layer. In Comparative Example 2, the non-woven fabric used is a spunlace non-woven fabric of 32 gsm. The stretchable laminate according to Comparative Example 2 is an ear manufactured by 3M Company. The stretchable laminate according to Comparative Example 2 was manufactured by laminating the spunlace non-woven fabric on both the main surfaces of the elastomer layer by affixing of melt-extrusions. A polyurethane-based thermoplastic elastomer of 42 gsm is used as the elastomer layer.

### Comparative Example 3

The stretchable laminate according to Comparative Example 3 is a stretchable laminate in which a non-woven fabric is laminated on both the main surfaces of the elastomer layer, being a small rectangular tab (manufactured by 3M Company). The stretchable laminate according to Comparative Example 3 was manufactured by laminating a spun-bonded non-woven fabric on both the main surfaces of the elastomer layer by heat lamination and annealing process. As the elastomer layer, a layer including a three-layer structure of skin layer-core layer-skin layer is used.

### Comparative Example 4

The stretchable laminate according to Comparative Example 4 is a stretchable laminate in which a non-woven fabric is laminated on both the main surfaces of the elastomer layer, being a small rectangular tab (manufactured by 3M Company). The stretchable laminate according to Comparative Example 4 was manufactured by laminating a spun-bonded non-woven fabric on both the main surfaces of the elastomer layer by heat lamination and annealing process. As the elastomer layer, a layer including a three-layer structure of skin layer-core layer-skin layer is used.

### Comparative Example 5

The stretchable laminate according to Comparative Example 5 is a stretchable laminate in which a non-woven fabric is laminated on both the main surfaces of the elastomer layer. The stretchable laminate according to Comparative Example 5 was manufactured by laminating a non-woven fabric on both the main surfaces of the elastomer layer by heat transfer bonding of a hot melt pressure sensitive adhesive. As the elastomer layer, a layer including a three-layer structure of skin layer-core layer-skin layer that has subjected to “activation” is used, and the hot melt pressure sensitive adhesive was applied by pattern coating.

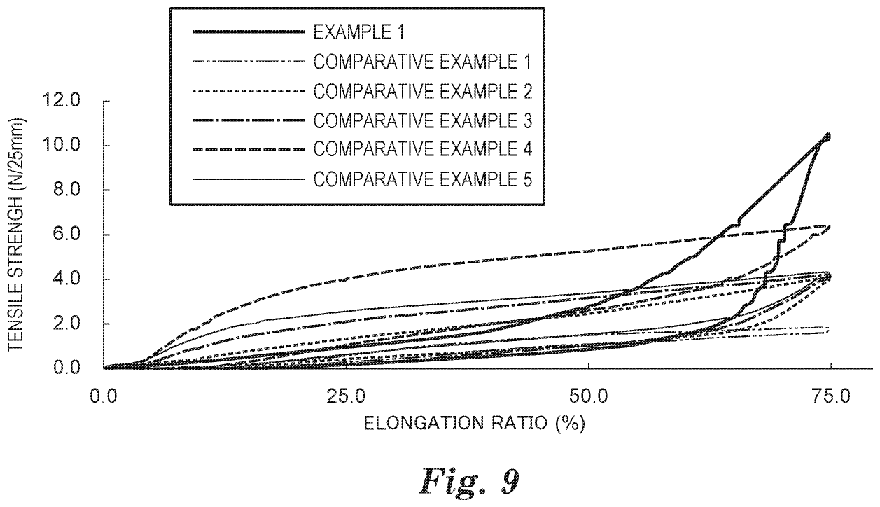

The results of the tensile test conducted on Example 1 and Comparative Examples 1 to 5 described above are shown in FIGS. 9 to 11 and Table 1 mentioned below. FIG. 9 is a graph showing the relationship between the CD elongation ratio and the tensile strength when the length of each of the stretchable laminates of Example 1 and Comparative Examples 1 to 5 in the CD is set to 15 mm and the length thereof in the MD is set to 25 mm. In Table 1 mentioned below, for the measurement of the tensile strength when the elongation ratio is 50% or 75%, the tensile speed is set to 300 mm/min, for example, as the tensile speed for attachment by the user (father, mother, or the like). Meanwhile, for the measurement of the maximum tensile strength and the maximum elongation ratio, the tensile speed is set to 100 mm/min as the tensile speed to examine the likelihood of tearing when the diaper is worn.

As shown in FIG. 9, it can be seen that the stretchable laminate including the knitted fabric according to Example 1 can be extended longer with a smaller tensile strength compared with different tabs of Comparative Examples 3 to 5. Further, it can be seen that the stretchable laminate according to Example 1 has a significantly higher tensile strength when the elongation ratio exceeds 70% than that of the stretchable laminates according to Comparative Examples 1 to 5. Specifically, as shown in Table 1, it can be seen that the stretchable laminate according to Example 1 has a tensile strength of less than or equal to 4.0 N/25 mm when the elongation ratio is 50% and can have an elongation ratio of less than or equal to 250% in stretching at 20 N/25 mm, and a low stress and a high strength have been achieved. Further, it is found that, in the stretchable laminate according to Example 1, the elongation ratio can be set to 50% at a tensile strength of less than or equal to 3.0 N/25 mm (2.8 N/25 mm) as well as the tensile strength when the elongation ratio was 75% can be greater than or equal to 10 N/25 mm (10.3 N/25 mm). In Table 1, the columns of the elongation ratio for 20 N/25 mm in Comparative Examples 1 and 2 are blank because breakage occurred and 20N is not achieved in Comparative Examples 1 and 2.

TABLE 1

| | Tensile stress (N/25 mm) | | | 20 N/25 mm | Maximum | Maximum |
|---|---|---|---|---|---|---|
| | Tensile | Tensile | Post tensile | elongation | tensile | elongation |
| | elongation | elongation | elongation | ratio | strength | ratio |
| | ratio 50% | ratio 75% | ratio 50% | (%) | (N/25 mm) | (%) |
| Example 1 | 2.8 | 10.3 | 0.9 | 96 | 40.1 | 171 |
| Comparative | 1.46 | 1.83 | 1.05 | — | 17.1 | 1050 |
| Example 1 | | | | | | |
| Comparative | 2.41 | 4.07 | 1.03 | — | 13.2 | 226 |
| Example 2 | | | | | | |
| Comparative | 3.11 | 4.22 | 0.97 | 316 | 33.7 | 540 |
| Example 3 | | | | | | |
| Comparative | 5.25 | 6.37 | 2.59 | 210 | 39.7 | 287 |
| Example 4 | | | | | | |
| Comparative | 3.34 | 4.32 | 1.51 | 546 | 23.6 | 686 |
| Example 5 | | | | | | |

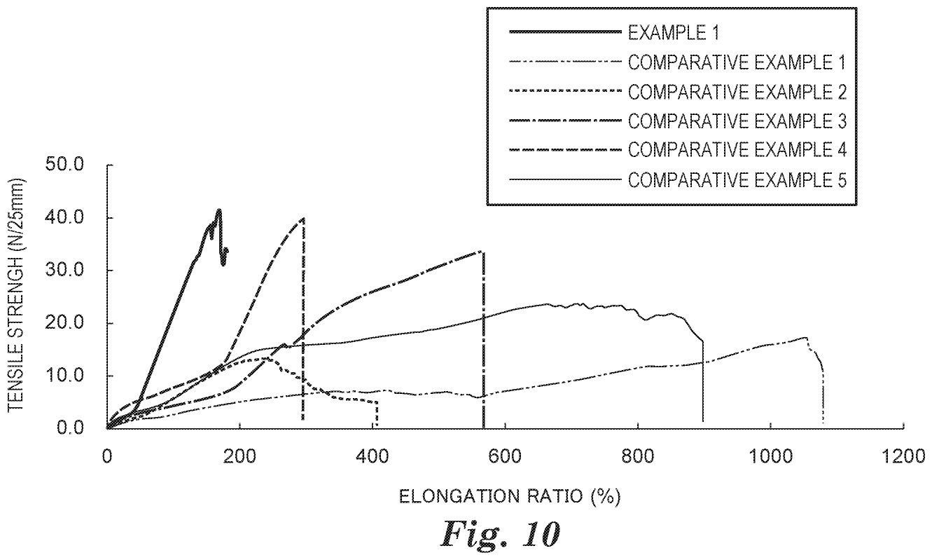

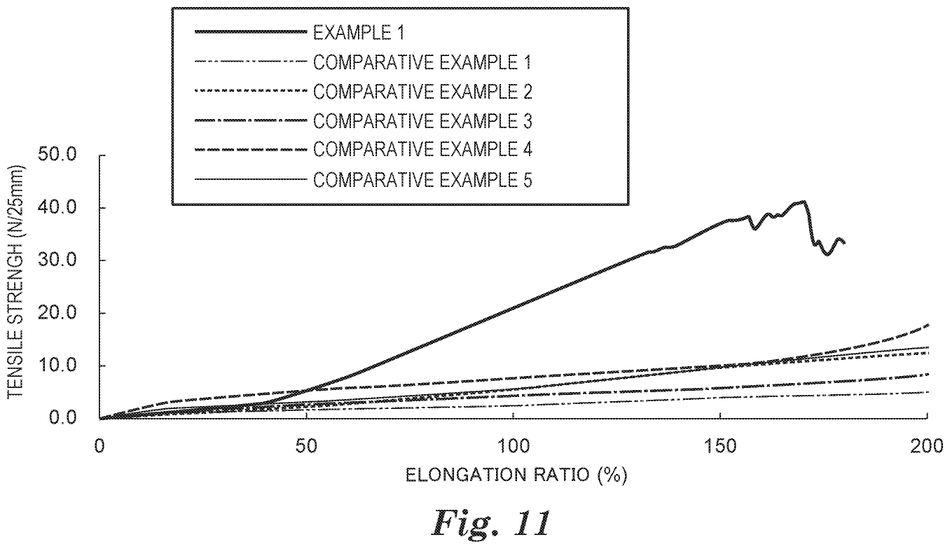

FIGS. 10 and 11 are graphs showing the relationship between the CD elongation ratio and the tensile strength when the length of each of the stretchable laminates of Example 1 and Comparative Examples 1 to 5 in the CD is set to 15 mm and the length thereof in the MD is set to 25 mm. As shown in FIGS. 10 and 11, it is found that the stretchable laminate including the knitted fabric according to Example 1 can enhance the tensile strength, in a state where the elongation ratio of the stretchable laminate is smaller than that of the stretchable laminates according to Comparative Examples 1 to 5. Further, it is found that the stretchable laminate according to Example 1 has a larger maximum value of the tensile strength than that of the stretchable laminates according to Comparative Examples 1 to 5. As shown in Table 1, the stretchable laminate according to Example 1 has a maximum elongation ratio of less than or equal to 250% (171%) and a maximum tensile strength of greater than or equal to 40 N/25 mm (40.1 N/25 mm). As described above, it is found that the stretchable laminate including the knitted fabric according to Example 1 can be easily extended at a lower stress as well as has a higher tensile strength as compared with the stretchable laminates of Comparative Examples 1 to 5.

Further, a tensile test was performed on the stretchable laminates of Example 1 and Examples 2 to 8 described below, and the tensile stress and the elongation ratio were measured. The tensile test was performed as described above.

### Example 2

In the stretchable laminate according to Example 2, a polyurethane-based elastomer of 42 gsm is used as the elastomer layer 13. In Example 2, the constituents other than
the elastomer layer 13 are the same as in the stretchable laminate according to Example 1.

### Example 3

In the stretchable laminate according to Example 3, as the knitted fabric 15, a weft-knitted (pearl stitch) fabric of polyester fiber of 150 gsm is used. In Example 3, the constituents other than the knitted fabric 15 are the same as in the stretchable laminate according to Example 1. The weight per unit area of the knitted fabric 15 (basis weight) is 150 g/m², the density of the knitted fabric 15 in the course direction is 50 yarns/inch, and the density of the knitted fabric 15 in the wale direction is 35 yarns/inch.

### Example 4

In the stretchable laminate according to Example 4, a polyurethane-based elastomer of 42 gsm is used as the elastomer layer 13, and a weft-knitted (pearl stitch) fabric of polyester fiber is used as the knitted fabric 15. In Example 4, the constituents other than the elastomer layer 13 and the knitted fabric 15 are the same as in the stretchable laminate according to Example 1. The weight per unit area of the knitted fabric 15 (basis weight) is 150 g/m², the density of the knitted fabric 15 in the course direction is 50 yarns/inch, and the density of the knitted fabric 15 in the wale direction is 35 yarns/inch.

### Example 5

In the stretchable laminate according to Example 5, as the knitted fabric 15, a warp-knitted (milanese stitch) fabric of polyester fiber of 100 gsm is used. In Example 5, the constituents other than the knitted fabric 15 are the same as in the stretchable laminate according to Example 1. The weight per unit area of the knitted fabric 15 (basis weight) is 100 g/m², the density of the knitted fabric 15 in the course direction is 32 yarns/inch, and the density of the knitted fabric 15 in the wale direction is 50 yarns/inch.

### Example 6

In the stretchable laminate according to Example 6, a polyurethane-based elastomer of 42 gsm is used as the elastomer layer 13, and a warp-knitted (milanese stitch) fabric of polyester fiber of 100 gsm is used as the knitted fabric 15. In Example 6, the constituents other than the elastomer layer 13 and the knitted fabric 15 are the same as in the stretchable laminate according to Example 1. The weight per unit area of the knitted fabric 15 (basis weight) is 100 g/m², the density of the knitted fabric 15 in the course direction is 32 yarns/inch, and the density of the knitted fabric 15 in the wale direction is 50 yarns/inch.

### Example 7

In the stretchable laminate according to Example 7, the adhesive layer 16 is formed by coating of 20 gsm of a urethane-based solvent. The constituents other than the adhesive layer 16 are the same as in the stretchable laminate according to Example 1.

### Example 8

In the stretchable laminate according to Example 8, a polyurethane-based elastomer of 42 gsm is used as the elastomer layer 13. Then, the adhesive layer 16 is formed by coating of 20 gsm of a urethane-based solvent. The constituents other than the elastomer layer 13 and the adhesive layer 16 are the same as in the stretchable laminate according to Example 1.

The results of the tensile test on the stretchable laminates according to Examples 1 to 8 described above are shown in Table 2. The contents of the experiments described in Table 2 are equivalent to the contents of the experiments described in Table 1.

TABLE 2

| | Tensile stress (N/25 mm) | | | 20 N/25 mm | Maximum | Maximum |
|---|---|---|---|---|---|---|
| | Tensile | Tensile | Post tensile | elongation | tensile | elongation |
| | elongation | elongation | elongation | ratio | strength | ratio |
| | ratio 50% | ratio 75% | ratio 50% | (%) | (N/25 mm) | (%) |
| Example 1 | 2.8 | 10.3 | 0.9 | 96 | 40.1 | 171 |
| Example 2 | 3.5 | 10.5 | 2.3 | 97 | 43.9 | 151 |
| Example 3 | 3.8 | 6.2 | 1.9 | 148 | 92.5 | 455 |
| Example 4 | 3.7 | 7.4 | 2.1 | 134 | 89.4 | 386 |
| Example 5 | 3.8 | 10.9 | 1.7 | 108 | 137 | 330 |
| Example 6 | 4.0 | 8.6 | 2.1 | 146 | 147 | 347 |
| Example 7 | 2.3 | 15.8 | 1.0 | 117 | 38.7 | 168 |
| Example 8 | 3.7 | 12.5 | 1.6 | 140 | 42.8 | 148 |

As shown in Table 2, it can be seen that any of the stretchable laminates according to Examples 1 to 8 has a tensile stress of less than or equal to 4.0 N/25 mm when the elongation ratio is 50% and can have an elongation ratio of less than or equal to 250% in stretching at 20 N/25 mm, and a low stress and a high strength have been achieved. Further, it is found that the stretchable laminates according to

Examples 1 and 7 can have an elongation ratio of 50% at a tensile strength of less than or equal to 3.0 N/25 mm. It is also found that the stretchable laminates according to Examples 1, 2, 5, 7, and 8 can have a tensile strength of greater than or equal to 10 N/25 mm when the elongation ratio is 75%. Hereinabove, it is found that the stretchable laminates according to Examples 1 to 8 can be easily extended at a low stress as well as have a high tensile strength.

Furthermore, when the stretchable laminates of Examples 1 to 8 are used for the tabs of diapers, the person who causes such a diaper to be worn (a guardian or the like in the case of a baby diaper) can lightly extend the tabs and, even if the diaper is slightly displaced, can attach the tab by extending the tab to a desired attachment position. It is also possible to reduce inconveniences such as temporary fixing, position adjustment, and reattachment. Further, due to the fact that the tabs can be appropriately stretched at a strength of 20 N, it is possible to obtain appropriate fittability and strength, suppress displacement of and leakage of the diaper, and reduce labor for cleaning up the leakage.

Further, in the case where the tabs have a tensile strength of less than or equal to 4.0 N/25 mm (for example, 3.0 N/25 mm) when the elongation ratio is 50%, the person who causes such a diaper to be worn (a guardian or the like) can lightly extend the stretchable laminate, and even if the diaper is slightly displaced, can easily attach the tabs to a desired attachment position. Further, since the tab can be extended and attached to a desired attachment position from the beginning, it is possible to reduce inconveniences such as temporary fixing, position adjustment, and reattachment.

Further, in the case where, with an elongation ratio of 75%, the stretchable laminate has a tensile strength 2 to 3 times the tensile strength when the elongation ratio is 50%, it is possible to make the person who causes such a diaper to be worn likely to recognize the degree of extension of the stretchable laminate (that the stretchable laminate can be extended to such a degree). Furthermore, in the case where the tensile strength when the elongation ratio after stretching is 50% is greater than or equal to 0.5 N/25 mm, the tightening can be appropriate for the wearer (an infant or the like) and the displacement during movement can be suppressed.

In the case where the elongation ratio in stretching at 20 N/25 mm is less than or equal to 250%, even if the waist circumference of the wearer changes due to turning in bed, abdomen bulging, or the like, it is possible to suppress breakage of the stretchable laminate and also no large deformation occurs. Thus the stretchable laminate contributes to prevention of displacement and leakage of the diaper. Accordingly, it is possible to reduce labor for cleaning up the leakage or the like.

In the case where the maximum tensile strength is greater than or equal to 20 N, the strength in the use of the stretchable laminate is sufficiently secured. Here, the “sufficient” strength means, for example, when a diaper is worn, such a strength that breakage does not occur even if the stretchable laminate (tabs or the like) is stretched while a diaper is placed under the buttocks of an infant or the like and the position of the diaper is adjusted. The maximum tensile strength is more preferably greater than or equal to 35 N. It is more preferable that the maximum elongation ratio be less than or equal to 500%, less than or equal to 400%, or less than or equal to 300% because the person who causes such a diaper to be worn may feel unreliable (the toughness is low) if the value is excessively large.

1. A stretchable laminate, comprising:
a non-woven fabric;
an elastomer layer laminated on the non-woven fabric; and
a knitted fabric laminated on the elastomer layer.

2. The stretchable laminate according to claim 1, comprising an adhesive layer interposed between the knitted fabric and the elastomer layer.

3. The stretchable laminate according to claim 2, wherein the adhesive layer is made of a pressure sensitive adhesive.

4. The stretchable laminate according to claim 2, wherein the adhesive layer includes an anchor coating material.

5. The stretchable laminate according to claim 2, wherein the adhesive layer is a film layer made by including a resin.

6. The stretchable laminate according to claim 1, wherein the density of the knitted fabric in the course direction is greater than or equal to 15 yarns/inch and less than or equal to 50 yarns/inch.

7. The stretchable laminate according to claim 1, wherein the density of the knitted fabric in the wale direction is greater than or equal to 15 yarns/inch and less than or equal to 50 yarns/inch.

8. The stretchable laminate according to claim 1, wherein the elongation ratio of the knitted fabric during elongation in at least one direction is greater than or equal to 150%.

9. A stretchable laminate, comprising:
a non-woven fabric;
an elastomer layer laminated on the non-woven fabric; and
a woven fabric laminated on the elastomer layer,
wherein at least a portion of a plurality of yarns composing the woven fabric are bonding-processed.

10. A locking member configured to be attached to a sanitary material, the locking member comprising:
a stretchable laminate including a knitted fabric laminated on an elastomer layer; and
a locking portion provided on an end portion of the stretchable laminate opposite to the sanitary material.

11. The locking member according to claim 10,
wherein the stretchable laminate includes a pair of low-stretchable portions,
one of the pair of low-stretchable portions is attached to the sanitary material, and
the locking portion is provided on the other of the pair of low-stretchable portions.

12. The locking member according to claim 10, comprising a pair of non-woven fabrics,
wherein the stretchable laminate includes a pair of low-stretchable portions respectively attached to each of the pair of non-woven fabrics,
one of the pair of non-woven fabrics is attached to the sanitary material, and
the locking portion is provided on the other of the pair of non-woven fabrics.

13. A locking member configured to be attached to a sanitary material, the locking member comprising:
a stretchable laminate of claim 9 being laminated on an elastomer layer and including a woven fabric, at least a portion of a plurality of yarns of the woven fabric being bonding-processed; and
a locking portion provided on an end portion of the stretchable laminate opposite to the sanitary material.

14. A sanitary material, comprising the locking member described in claim 10 as a tab.
